(19) United States
(12) Patent Application Publication
(10) Pub. No.: US 2005/0125033 A1
(43) Pub. Date: Jun. 9, 2005

(54) Wound closure apparatus

(76) Inventors: Karen M. McNally-Heintzelman, Cambridge, MA (US); Mark T. Duffy, Green Bay, WI (US); Jeffrey N. Bloom, Chicago, IL (US); Douglas L. Heintzelman, Cambridge, MA (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/003,648

(22) Filed: Dec. 3, 2004

Related U.S. Application Data
(60) Provisional application No. 60/526,819, filed on Dec. 4, 2003.

(51) Int. Cl. A61B 17/08; A61D 1/00

(52) U.S. Cl. 606/213; 606/205

(57) ABSTRACT

A wound closure apparatus is provided. The apparatus includes a pair of clamp arms, and a pair of blades, each having a gripping surface, coupled to the clamp arm.

### BACKGROUND

Since their first introduction on the U.S. market, tissue adhesives have been gaining acceptance in the operating room as an attractive alternative to the use of sutures for closing skin lacerations. This technology takes less time to apply and causes less pain than suturing, and requires no dressing or needle use. This is especially useful in the case of children, or when small-caliber sutures must be removed from sensitive areas of the face. In addition, the risk of needle-stick injury and transmissible infections for operating room personnel is removed.

Surgical adhesives such as cyanoacrylate glues have the advantage that they are generally easy to dispense. However, application of adhesives during any surgical procedure can be cumbersome. Because of their liquid nature, these adhesives are difficult to precisely position on tissue and thus require adept and delicate application if precise positioning is desired. Cyanoacrylates also harden rapidly; therefore, the time available to the surgeon for proper tissue alignment is limited.

Improvements to currently available biologic and synthetic adhesives are addressed in a co-pending U.S. patent application: Non-Light Activated Biological Adhesive Device, System, and Methods of Use Thereof, Ser. No. 10/610,068, filed June 2003 (to co-inventors McNally-Heintzelman K M, Heintzelman D L, Bloom J N and Duffy M T). The present application describes locking wound closure clamps designed to facilitate the use of the adhesive described in the above mentioned U.S. patent application, as well as other surgical adhesives and adhesive-enhanced repair techniques.

### SUMMARY

One aspect of the present invention relates to a locking wound closure apparatus, system and method for placing wound edges in close apposition to allow for a proper closure. In accordance with the present invention, a wound closure apparatus is provided, which includes first and second clamp arms coupled together at one end. Each clamp arm includes a blade. Each blade includes a gripping surface. The blades on the clamp are separated by a well for placement of an adhesive material. The clamp is configured to isolate the wound field and align the wound edges to allow for precise placement of the adhesive material.

Another aspect of the present invention relates to a locking mechanism that allows a single user to both clamp and set an adhesive material in place. Thus, in an alternative embodiment, the wound closure apparatus further includes a locking mechanism. In the illustrated embodiment, the locking mechanism includes a pair of spaced apart locking members configured to engage one another. In the illustrated embodiment, a first locking member is coupled to the first clamp arm and a second locking member is coupled to the second clamp arm. In certain embodiments, the locking mechanism has a variable width to allow for customized selection of the size of the adhesive material.

Yet another aspect of the present invention relates to a plurality of interchangeable blades, manufactured, for example, with variable length and weight, to suit a broad range of wound closures. Accordingly, a wound closure system is also provided, which includes a wound closure apparatus and a plurality of blades.

Still another aspect of the present invention involves a separate instrument to provide custom curvilinear bending of the blades such that they can be contoured to suit non-linear wounds. Accordingly, an alternative wound closure system is provided, which includes a wound closure apparatus and a blade adjustment apparatus.

A further aspect of the present invention relates to a locking wound closure apparatus configured to facilitate closure of higher tension wounds. In one embodiment, the clamp of the wound closure apparatus has a heavier weight. In another embodiment, the blades include dentals or dental-like protrusions configured to engage thicker skin and/or skin under high tension.

### BRIEF DESCRIPTION OF THE DRAWINGS

FIG. 1a is a perspective view of an embodiment of a wound closure apparatus used to place wound edges in close apposition for proper closure;

FIG. 1b is a side view of a first embodiment of a blade and gripping portion for use with the wound closure apparatus of FIG. 1a;

FIG. 1c is a side view of a second embodiment of a blade and gripping portion for use with the wound closure apparatus of FIG. 1a;

FIG. 1d is a top view of the apparatus of FIG. 1a, while in use holding a wound closed;

FIG. 1e is a side view of a third embodiment of a blade and gripping portion for use with the apparatus of FIG. 1a;

FIG. 1f is a top view of the apparatus of FIG. 1a, with the blade and gripping portion of FIG. 1e, while in use;

FIG. 2a is a perspective view of a first alternative embodiment of a wound closure apparatus used to facilitate closure of high tension wounds;

FIG. 2b is a perspective view of a second alternative embodiment of a wound closure apparatus used to facilitate closure of high tension wounds;

FIG. 2c is a perspective view of a third embodiment of a wound closure apparatus used to facilitate closure of high tension wounds;

FIG. 2d is a top view of the blades of the apparatus of FIG. 2a in use, closing around an incision; and

FIG. 2e is a side view of the blade shown in FIG. 2a.

### DETAILED DESCRIPTION

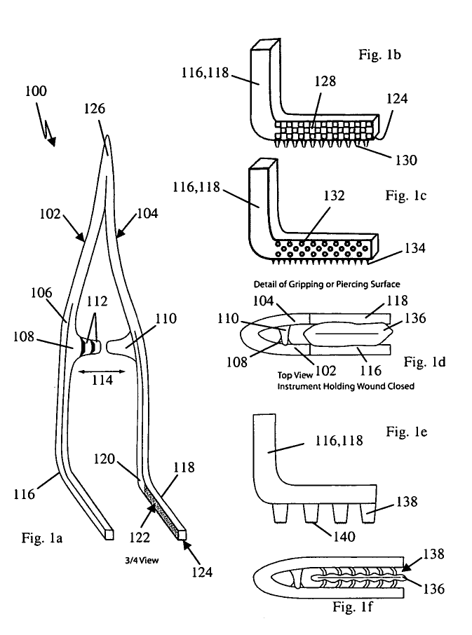

FIGS. 1a-1f show various features and alternative designs of portions of a wound closure apparatus in accordance with the present invention. FIG. 1a shows one embodiment of the wound closure apparatus.

The wound closure apparatus 100 of FIG. 1a includes first and second clamp arms 102, 104 coupled together at a joint 126 by an adhesive, solder, fastener, or any other suitable coupling means known in the art. Clamp arms 102, 104 include locking members 108, 110 of locking mechanism 106. Clamp arms 102, 104 also include blade portions 116, 118 located on the ends of clamp arms 102, 104 opposite joint 126.

The various portions of wound closure apparatus 100 are made of surgical steel or other suitable material known in the art. Portions of clamp arms 102, 104 may be coated or enclosed in a synthetic material such as a foam, rubber or other suitable high-friction material to provide for easier maintenance and handling by the medical professional.

Locking mechanism 106 operates to hold blades 116, 118 in place after application to a wound area. In particular, locking mechanism 106 allows the medical professional to remove his/her hand from wound closure apparatus 100 after a wound has been closed, so that the hand is free to apply an adhesive to the wound site or perform other tasks.

First locking member 108 includes at least one coupling portion 112, e.g., gradation, ridge, hook, or the like that is sized to mate with a corresponding coupling portion (not shown), e.g., depressions, catches, rings, or the like of second locking member 110. It is understood that any suitable coupling mechanism known in the art may be used to couple locking members 108, 110 together.

In addition, locking members 108, 110 preferably include at least two such coupling mechanisms, so that wound closure apparatus 100 is adjustable to wounds of varying widths or thicknesses.

FIG. 1d shows an example of wound closure apparatus 100 in a locked position, holding a wound closed with the wound edges aligned. Through the use of an adjustable locking mechanism 106 and various alternative blades 116, 118, the amount of force or tension exerted on the wound site can be controlled and adjusted to achieve the desired alignment of the wound edges.

As noted above, blades 116, 118 are configured with a gripping portion 122. The characteristics of the gripping portion are selected as appropriate based on the type, size, or location of the wound, the type of adhesive or method of wound closure being used, or other criteria.

FIG. 1b shows one variation of a gripping portion. Interior surface 128 of at least one of blades 116, 118 has an irregular character, for example, including alternating raised areas (shown as shaded squares) and flat or depressed areas (unshaded squares). Protrusions 130 are coupled to outer surface 124 of blades 116, 118 by solder, adhesive or other coupling means, or are molded with each blade 116, 118. As shown in FIG. 1b, protrusions 130 are essentially cone-like in shape.

FIG. 1c shows another variation of gripping portion 122, including bumps 132 coupled thereto in an alternating pattern to provide an irregular surface. Bumps 132 are coupled to gripping portion 122 via any suitable coupling means mentioned above or otherwise known in the art, or are molded into gripping portion 122 by known techniques. Protrusions 134 generally have a smaller diameter or thickness than the protrusions 130 shown in FIG. 1b and are essentially pin- or needle-like in shape.

In the alternative design of FIG. 1e, gripping portion 122 is more or less smooth, but dentals or other tooth-like protrusions 138 are coupled to outer surface 124. As shown, each protrusion 138 has an irregular edge 140. FIG. 1f shows the embodiment of FIG. 1e, in use to hold wound 136 closed.

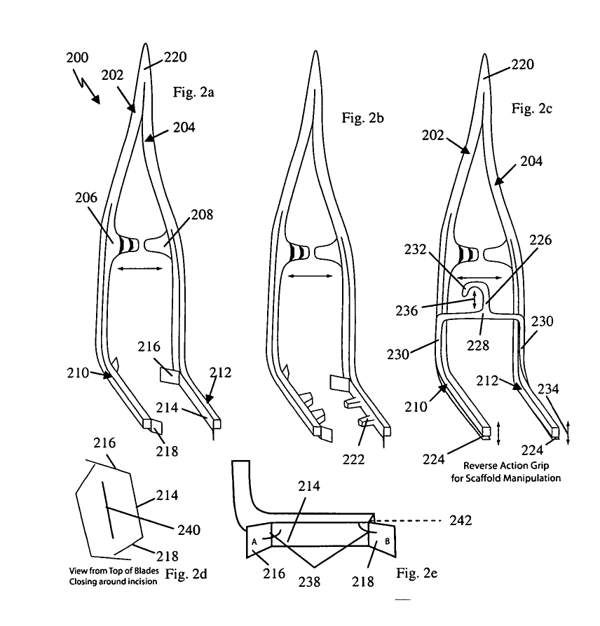

FIGS. 2a, 2b, and 2c show alternative embodiments of a wound closure apparatus 200 in accordance with the present invention.

As shown in FIG. 2a, wound closure apparatus 200 includes clamp arms 202, 204, blades 210, 212 coupled to or molded from clamp arms 202, 204, and locking members 206, 208 as described above. Clamp arms 202, 204 are coupled together at end 220.

In the embodiment of FIG. 2a, blades 210, 212 each have a gripping member which includes a center portion 214 and end portions 216, 218 adjacent each end of center portion 214 as shown. End portions 216, 218 are angled inwardly away from the blades 210, 212 to which they are attached. In the illustrated embodiment, the angles 238 formed by the intersection of the end portions 216, 218 with a longitudinal axis 242 of the clamp arm is greater than 90° as best shown in FIG. 2e.

The inner surfaces of end portions 216, 218 and center portion 214, i.e., the surfaces facing toward the gripping surface of the opposite clamp arm, are illustratively smooth. However, it is understood that one or more of such inner surfaces may have an irregular or rough character. For example, FIG. 2b shows a center portion 214 with at least one dental-like protrusion 222.

End portions 216, 218 are configured to form a “well” around the wound when apparatus 200 is in use. The well is designed to hold a volume of adhesive in the area of the wound.

The embodiment of FIG. 2c includes an alternative form of gripping mechanism. This embodiment is configured to enable the medical professional to position, move or manipulate a scaffolding or adhesive composite while still holding the edges of a wound in the desired alignment.

The alternative gripping mechanism of FIG. 2c, referred to as the “reverse action grip,” includes an adjustment member 226 coupled to or molded with a cross member 228. Cross member 228 is coupled to or molded with side members 230 which run along the outer edges of clamp arms 202, 204.

Adjustment member 226 includes a curved portion 232 which controls movement of reverse action grips 224. When pressure is applied to adjustment member 226 in the direction of arrow 236, reverse action grips 224 move in the corresponding direction of arrow 234. For example, if adjustment member 226 is moved downwardly toward blades 210, 212, reverse action grips 224 will move downwardly away from blades 210, 212, thus creating a gap between grips 224 and blades 210, 212. If adjustment member 226 is moved upwardly toward end 220, reverse action grips 224 move upwardly toward blade portions 210, 212, thus closing the gap between grips 224 and blades 210, 212. Operation of adjustment member 226 can be performed independently of locking mechanism 206, 208. In the illustrated embodiment, adjustment member 226 controls the action of both grips 224 simultaneously. In an alternative embodiment, grips 224 are independently controllable.

In the illustrated embodiment, cross member 228 is sufficiently flexible or resilient so that when clamp arms 202, 204 move toward or away from each other (e.g., to engage locking mechanism 206, 208), cross member 228 flexes inwardly or outwardly as needed.

In FIG. 2c, curved portion 232 is sized to accommodate a human finger, such as an index finger, for ease of use by the medical professional. With end 220 proximate to the palm of the hand, and the index finger curled around curved portion 232, the action of retracting index finger backwardly toward the palm accomplishes upwardly movement of reverse action grips 224 toward blades 210, 212.

FIG. 2d shows the embodiment of FIG. 2a while in use to surround a wound site 240 and keep the wound edges aligned.

Although the present invention has been described in detail with reference to certain exemplary embodiments, it is understood that variations and modifications exist and are within the scope and spirit of the present invention.

1. A wound closure apparatus, comprising:
a first clamp arm,
a second clamp arm coupled to the first clamp arm,
first and second blades coupled to the first and second clamp arms, respectively, and
first and second gripping portions coupled to the first and second blades, respectively, wherein the blades are spaced apart to define a well for placement of an adhesive material.

2. The apparatus of claim 1, further comprising a locking mechanism.

3. The apparatus of claim 2, wherein the locking mechanism includes a pair of spaced part locking members configured to engage one another.

4. The apparatus of claim 3, wherein the locking mechanism includes a first locking member coupled to the first clamp arm and a second locking member coupled to the second clamp arm.

5. The apparatus of claim 3, wherein the locking mechanism has a variable width.

6. A system including the apparatus of claim 1 and a plurality of interchangeable blades.

7. The system of claim 6, wherein the blades have a variable length to suit a broad range of wound closures.

8. The system of claim 6, wherein the blades have a variable weight to suit a broad range of wound closures.

9. The system of claim 6, further comprising means for bending the blades to accommodate non-linear wounds.

10. The system of claim 6, further comprising a blade adjustment apparatus.

11. The apparatus of claim 1, wherein the blades include at least one dental-like protrusion.

12. The apparatus of claim 1, wherein at least one of the gripping portions has an irregular surface.

13. The apparatus of claim 1, further comprising a pair of reverse-action grips.

14. A wound closure apparatus, comprising:
a pair of clamp arms coupled together at a first end,
each clamp arm having a blade portion coupled thereto at a second end spaced apart from the first end, and
means coupled to the blade portions for aligning wound edges for close apposition.
